(19) United States
(12) Patent Application Publication
(10) Pub. No.: US 2011/0207773 A1
(43) Pub. Date: Aug. 25, 2011

(54) NOVEL PHENYL-QUINOLINE-CARBOXYLIC ACID PYRIDINE DERIVATIVES USEFUL AS MODULATORS OF NICOTINIC ACETYLCHOLINE RECEPTORS

(75) Inventors: Antonio Nardi, Herzogenrath (DE); Jeppe Kejser Christensen, Kovenhavn N (DK); Dan Peters, Malmo (SE); Tino Dyhring, Solrod (DK)

(73) Assignee: NEUROSEARCH A/S, Ballerup (DK)

(21) Appl. No.: 13/059,799

(22) PCT Filed: Aug. 20, 2009
(86) PCT No.: PCT/EP2009/060768

§ 371 Date: May 5, 2011

(30) Foreign Application Priority Data
Aug. 21, 2008 (DK) PA 2008 01140

(51) Int. Cl. A61K 31/4709; C07D 401/12; C07D 405/14; A61P 25/00; A61P 25/22; A61P 25/28; A61P 25/16; A61P 25/24; A61P 25/18; A61P 3/04; A61P 25/20; A61P 25/02; A61P 25/14; A61P 25/08; A61P 15/00; A61P 9/12; A61P 9/06; A61P 21/00; A61P 1/12; A61P 11/06; A61P 5/00; A61P 25/04; A61P 29/00; A61P 17/00; A61P 17/10; A61P 25/30; A61P 25/36

(52) U.S. Cl. 514/314; 546/169

(57) ABSTRACT

This invention relates to novel phenyl-quinoline-carboxylic acid pyridine derivatives, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### TECHNICAL FIELD

This invention relates to novel phenyl-quinoline-carboxylic acid pyridine derivatives, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### BACKGROUND ART

The endogenous cholinergic neurotransmitter, acetylcholine, exert its biological effect via two types of cholinergic receptors, the muscarinic Acetyl Choline Receptors (mAChR) and the nicotinic Acetyl Choline Receptors (nAChR).

As it is well established that muscarinic acetylcholine receptors dominate quantitatively over nicotinic acetylcholine receptors in the brain area important to memory and cognition, and much research aimed at the development of agents for the treatment of memory related disorders have focused on the synthesis of muscarinic acetylcholine receptor modulators.

Recently, however, an interest in the development of nAChR modulators has emerged. Several diseases are associated with degeneration of the cholinergic system i.e. senile dementia of the Alzheimer type, vascular dementia and cognitive impairment due to the organic brain damage disease related directly to alcoholism.

### SUMMARY OF THE INVENTION

The present invention is devoted to the provision novel modulators of the nicotinic receptors, which modulators are useful for the treatment of diseases or disorders related to the cholinergic receptors, and in particular the nicotinic acetylcholine α7 receptor subtype.

The compounds of the invention may also be useful as diagnostic tools or monitoring agents in various diagnostic methods, and in particular for in vivo receptor imaging (neuroimaging), and they may be used in labelled or unlabelled form.

In its first aspect the invention provides phenyl-quinoline-carboxylic acid pyridine derivatives of Formula I

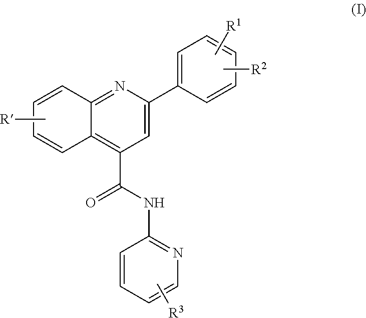

a stereoisomer thereof or a mixture of its stereoisomers, or a pharmaceutically acceptable salt thereof, wherein

R′ represents hydrogen or halo;

R¹ and R², independently of each other, represent a substituent selected from the group consisting of hydrogen, halo, trifluoromethyl, trifluoromethoxy, nitro, cyano, hydroxy and alkoxy; or R¹ and R² together represents a methylenedioxy or ethylenedioxy group; and

R³ represents hydrogen, halo, nitro or alkyl.

In a second aspect the invention provides pharmaceutical compositions comprising a therapeutically effective amount of the phenyl-quinoline-carboxylic acid pyridine derivative of the invention, or a pharmaceutically acceptable addition salt thereof, together with at least one pharmaceutically acceptable carrier or diluent.

Viewed from another aspect the invention relates to the use of the phenyl-quinoline-carboxylic acid pyridine derivative of the invention, or a pharmaceutically acceptable addition salt thereof, for the manufacture of pharmaceutical compositions/medicaments for the treatment, prevention or alleviation of a disease or a disorder or a condition of a mammal, including a human, which disease, disorder or condition is responsive to modulation of cholinergic receptors.

In yet another aspect the invention provides a method for treatment, prevention or alleviation of diseases, disorders or conditions of a living animal body, including a human, which disorder, disease or condition is responsive to modulation of cholinergic receptors, and which method comprises the step of administering to such a living animal body in need thereof a therapeutically effective amount of the phenyl-quinoline-carboxylic acid pyridine derivative of the invention.

Other objects of the invention will be apparent to the person skilled in the art from the following detailed description and examples.

### DETAILED DISCLOSURE OF THE INVENTION

### Phenyl-quinoline-carboxylic acid pyridine derivatives

In its first aspect the invention provides phenyl-quinoline-carboxylic acid pyridine derivatives of Formula I

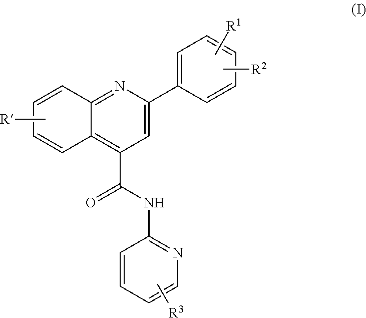

a stereoisomer thereof or a mixture of its stereoisomers, or a pharmaceutically acceptable salt thereof, wherein

R′ represents hydrogen or halo;

R¹ and R², independently of each other, represent a substituent selected from the group consisting of hydrogen, halo, trifluoromethyl, trifluoromethoxy, nitro, cyano, hydroxy and alkoxy; or R¹ and R² together represents a methylenedioxy or ethylenedioxy group; and

R³ represents hydrogen, halo, nitro or alkyl;

provided, however,

that R¹, R² and R³ cannot all represent hydrogen; and

if R¹ and R² both represent hydrogen, then R³ cannot represent chloro or nitro or methyl; and

if R¹ and R² both represent chloro or methoxy, then R³ cannot represent hydrogen, chloro, bromo, nitro or methyl; and

if one of R¹ and R² represents halo or alkoxy, and the other of R¹ and R² represents hydrogen, then R³ cannot represent hydrogen, chloro, nitro or methyl.

In a preferred embodiment the phenyl-quinoline-carboxylic acid pyridine derivative of the invention is a compound of Formula I

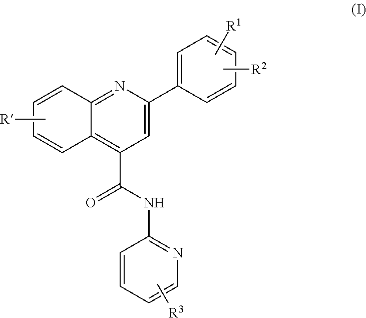

a stereoisomer thereof or a mixture of its stereoisomers, or a pharmaceutically acceptable salt thereof, wherein

R′ represents hydrogen or halo;

R¹ and R², independently of each other, represent a substituent selected from the group consisting of hydrogen, halo, trifluoromethyl, trifluoromethoxy, nitro, cyano, hydroxy and alkoxy; and

R³ represents hydrogen, halo, nitro or alkyl.

In another preferred embodiment the phenyl-quinoline-carboxylic acid pyridine derivative of the invention is a compound of Formula Ia

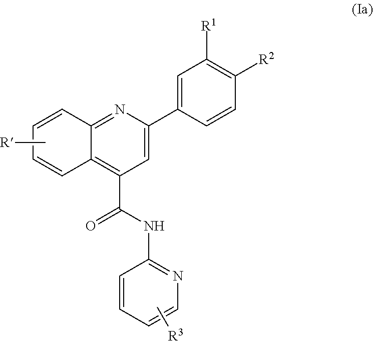

a stereoisomer thereof or a mixture of its stereoisomers, or a pharmaceutically acceptable salt thereof, wherein R′, R¹, R² and R³ are as defined above.

In a third preferred embodiment the phenyl-quinoline-carboxylic acid pyridine derivative of the invention is a compound of Formula Ib

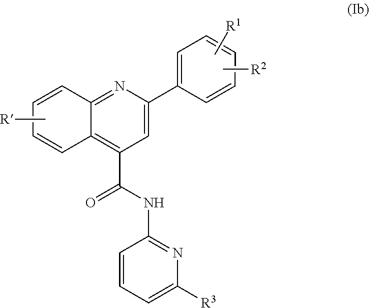

a stereoisomer thereof or a mixture of its stereoisomers, or a pharmaceutically acceptable salt thereof, wherein R′, R¹, R² and R³ are as defined above.

In a fourth preferred embodiment the phenyl-quinoline-carboxylic acid pyridine derivative of the invention is a compound of Formula Ic

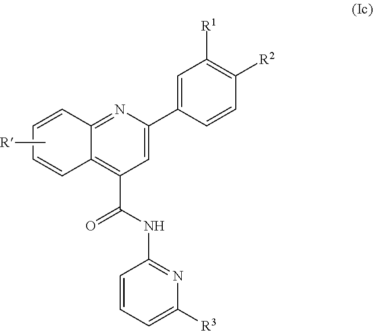

a stereoisomer thereof or a mixture of its stereoisomers, or a pharmaceutically acceptable salt thereof, wherein R′, R¹, R² and R³ are as defined above.

In a fifth preferred embodiment the phenyl-quinoline-carboxylic acid pyridine derivative of the invention is a compound of Formula I, Ia, Ib or Ic, a stereoisomer thereof or a mixture of its stereoisomers, or a pharmaceutically acceptable salt thereof, wherein R′ represents hydrogen or halo.

In a more preferred embodiment R′ represents hydrogen.

In another more preferred embodiment R′ represents halo, and in particular fluoro.

In a sixth preferred embodiment the phenyl-quinoline-carboxylic acid pyridine derivative of the invention is a compound of Formula I, Ia, Ib or Ic, a stereoisomer thereof or a mixture of its stereoisomers, or a pharmaceutically acceptable salt thereof, wherein R¹ and R², independently of each other, represent a substituent selected from the group consisting of hydrogen, halo, trifluoromethyl, trifluoromethoxy, nitro, cyano, hydroxy and alkoxy; or R¹ and R² together represents a methylenedioxy or ethylenedioxy group.

In a seventh preferred embodiment the phenyl-quinoline-carboxylic acid pyridine derivative of the invention is a compound of Formula I, Ia, Ib or Ic, a stereoisomer thereof or a mixture of its stereoisomers, or a pharmaceutically acceptable salt thereof, wherein R¹ and R², independently of each other, represent a substituent selected from the group consisting of hydrogen, halo, trifluoromethyl, trifluoromethoxy, nitro, cyano, hydroxy and alkoxy.

In a more preferred embodiment R¹ and R² both represent halo, trifluoromethyl, trifluoromethoxy, nitro, cyano, hydroxy or alkoxy.

In another more preferred embodiment R¹ and R² both represent halo, and in particular chloro, or alkoxy, and in particular methoxy.

In a third more preferred embodiment R¹ represents hydrogen; and R² represents halo, trifluoromethyl, trifluoromethoxy, nitro, cyano, hydroxy or alkoxy.

In a fourth more preferred embodiment R¹ represents hydrogen; and R² represents halo, and in particular chloro, hydroxy or alkoxy, and in particular methoxy.

In a fifth more preferred embodiment R¹ represents hydrogen, halo, trifluoromethyl, trifluoromethoxy, nitro, cyano, hydroxy or alkoxy.

In a sixth more preferred embodiment R¹ represents hydrogen, halo, and in particular chloro, or alkoxy, and in particular methoxy.

In a seventh eight more preferred embodiment R² represents halo, trifluoromethyl, trifluoromethoxy, nitro, cyano, hydroxy or alkoxy.

In an eight more preferred embodiment R² represents halo, and in particular chloro or bromo, hydroxy or alkoxy, and in particular methoxy.

In an eight preferred embodiment the phenyl-quinoline-carboxylic acid pyridine derivative of the invention is a compound of Formula I, Ia, Ib or Ic, a stereoisomer thereof or a mixture of its stereoisomers, or a pharmaceutically acceptable salt thereof, wherein R¹ and R² together represents a methylenedioxy or ethylenedioxy group.

In a more preferred embodiment R¹ and R² together represents a methylenedioxy group.

In another more preferred embodiment R¹ and R² together represents an ethylenedioxy group.

In a ninth preferred embodiment the phenyl-quinoline-carboxylic acid pyridine derivative of the invention is a compound of Formula I, Ia, Ib or Ic, a stereoisomer thereof or a mixture of its stereoisomers, or a pharmaceutically acceptable salt thereof, wherein R³ represents hydrogen, halo, nitro or alkyl.

In a more preferred embodiment R³ represents hydrogen.

In another more preferred embodiment R³ represents halo, and in particular fluoro or chloro.

In a third more preferred embodiment R³ represents nitro.

In a fourth more preferred embodiment R³ represents alkyl, and in particular methyl.

In a most preferred embodiment the phenyl-quinoline-carboxylic acid pyridine derivative of the invention is

2-(3,4-Dichloro-phenyl)-quinoline-4-carboxylic acid (6-fluoro-pyridin-2-yl)-amide;

2-(4-Chloro-phenyl)-quinoline-4-carboxylic acid (6-fluoro-pyridin-2-yl)-amide;

2-(3,4-Dichloro-phenyl)quinoline-4-carboxylic acid (5-fluoro-pyridin-2-yl)-amide;

2-(4-Methoxy-phenyl)-quinoline-4-carboxylic acid (6-fluoro-pyridin-2-yl)-amide;

2-(3,4-Dimethoxy-phenyl)-quinoline-4-carboxylic acid (6-fluoro-pyridin-2-yl)-amide;

2-(4-Hydroxy-phenyl)-quinoline-4-carboxylic acid (6-fluoro-pyridin-2-yl)-amide;

2-(4-Bromo-phenyl)-quinoline-4-carboxylic acid (5-methyl-pyridin-2-yl)-amide;

8-Fluoro-2-(4-methoxy-phenyl)-quinoline-4-carboxylic acid (6-fluoro-pyridin-2-yl)-amide;

8-Fluoro-2-(4-hydroxy-phenyl)-quinoline-4-carboxylic acid (6-fluoro-pyridin-2-yl)-amide;

6-Fluoro-2-(4-methoxy-phenyl)-quinoline-4-carboxylic acid (6-fluoro-pyridin-2-yl)-amide;

6-Fluoro-2-(4-hydroxy-phenyl)-quinoline-4-carboxylic acid (6-fluoro-pyridin-2-yl)-amide;

2-Benzo[1,3]dioxo-5-yl-quinoline-4-carboxylic acid (6-fluoro-pyridin-2-yl)-amide;

2-(2,3-Dihydro-benzo[1,4]dioxin-6-yl)quinoline-4-carboxylic acid (6-fluoro-pyridin-2-yl)-amide; or

7-Fluoro-2-(4-methoxy-phenyl)-quinoline-4-carboxylic acid (6-fluoro-pyridin-2-yl)-amide;

a stereoisomer thereof or a mixture of its stereoisomers, or a pharmaceutically acceptable salt thereof.

Any combination of two or more of the embodiments described herein is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkoxy group designates an “alkyl-O-” group, wherein alkyl is as defined above. Examples of preferred alkoxy groups of the invention include methoxy and ethoxy.

### Pharmaceutically Acceptable Salts

The phenyl-quinoline-carboxylic acid pyridine derivative of the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Metal salts of a phenyl-quinoline-carboxylic acid pyridine derivative of the invention include alkali metal salts, such as the sodium salt of a compound of the invention containing a carboxy group.

### Steric Isomers

It will be appreciated by those skilled in the art that the phenyl-quinoline-carboxylic acid pyridine derivatives of the present invention may exist in different stereo isomeric forms, including enantiomers, diastereomers, as well as geometric isomers (cis-trans isomers). The invention includes all such stereoisomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optically active starting materials or intermediates.

### Methods of Producing Phenyl-Quinoline-Carboxylic Acid Pyridine Derivatives

The phenyl-quinoline-carboxylic acid pyridine derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The present invention is devoted to the provision novel modulators of the nicotinic receptors, which modulators are useful for the treatment of diseases or disorders related to the cholinergic receptors, and in particular the nicotinic acetylcholine receptor (nAChR). Preferred compounds of the invention show a pronounced nicotinic acetylcholine α7 receptor subtype selectivity.

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

The compounds of the invention may also be useful as diagnostic tools or monitoring agents in various diagnostic methods, and in particular for in vivo receptor imaging (neuroimaging), and they may be used in labelled or unlabelled form.

In a preferred embodiment the disease, disorder or condition contemplated according to the invention, and responsive to modulation of nicotinic acetylcholine receptors is anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, peripheral neuropathy, autism, dyslexia, tardive dyskinesia, hyperkinesia, epilepsy, post-traumatic syndrome, social phobia, a sleeping disorder, pseudo dementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhea, asthma, epilepsy, tardive dyskinesia, hyperkinesia, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to postherpetic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated with withdrawal symptoms caused by termination of use of addictive substances, including nicotine withdrawal symptoms, opioid withdrawal symptoms including heroin, cocaine and morphine, benzodiazepine withdrawal symptoms including benzodiazepine-like drugs and alcohol.

In a more preferred embodiment the disease, disorder or condition responsive to modulation of nicotinic acetylcholine receptors is a cognitive disorder, psychosis, schizophrenia or depression.

In another more preferred embodiment the disease, disorder or condition responsive to modulation of nicotinic acetylcholine receptors is associated with smooth muscle contractions, including convulsive disorders, angina pectoris, premature labour, convulsions, diarrhoea, asthma, epilepsy, tardive dyskinesia, hyperkinesia, premature ejaculation and erectile difficulty.

In still another more preferred embodiment the disease, disorder or condition responsive to modulation of nicotinic acetylcholine receptors is related to the endocrine system, such as thyrotoxicosis and pheochromocytoma.

In yet another more preferred embodiment the disease, disorder or condition responsive to modulation of nicotinic acetylcholine receptors is a neurodegenerative disorder including transient anoxia and induced neuro-degeneration.

In a further more preferred embodiment the disease, disorder or condition responsive to modulation of nicotinic acetylcholine receptors is pain, including mild, moderate or even severe pain of acute, chronic or recurrent character, as well as pain caused by migraine, postoperative pain, and phantom limb pain. The pain may in particular be neuropathic pain, chronic headache, central pain, pain related to diabetic neuropathy, to postherpetic neuralgia, or to peripheral nerve injury.

In a further more preferred embodiment the disease, disorder or condition responsive to modulation of nicotinic acetylcholine receptors is an inflammatory skin disorder such as acne and rosacea, Crohn's disease, inflammatory bowel disease, ulcerative colitis, and diarrhoea.

Finally the compounds of the invention may be useful for the treatment of withdrawal symptoms caused by termination of use of addictive substances. Such addictive substances include nicotine containing products such as tobacco, opioids such as heroin, cocaine and morphine, benzodiazepines and benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

In this context “treatment” covers treatment, prevention, prophylactics and alleviation of withdrawal symptoms and abstinence as well as treatment resulting in a voluntary diminished intake of the addictive substance.

### Pharmaceutical Compositions

In another aspect the invention provides novel pharmaceutical compositions comprising a therapeutically effective amount of phenyl-quinoline-carboxylic acid pyridine derivative of the invention.

While a phenyl-quinoline-carboxylic acid pyridine derivative of the invention for use in therapy may be administered in the form of the raw compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the phenyl-quinoline-carboxylic acid pyridine derivative of the invention, or a pharmaceutically acceptable salt or derivative thereof, together with one or more pharmaceutically acceptable carriers therefore, and, optionally, other therapeutic and/or prophylactic ingredients know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

The phenyl-quinoline-carboxylic acid pyridine derivatives of the present invention are valuable nicotinic receptor modulators, and therefore useful for the treatment of a range of ailments involving cholinergic dysfunction as well as a range of disorders responsive to the action of nAChR modulators.

In another aspect the invention provides a method for the treatment, prevention or alleviation of a disease or a disorder or a condition of a living animal body, including a human, which disease, disorder or condition is responsive to modulation of cholinergic receptors, and which method comprises administering to such a living animal body, including a human, in need thereof an effective amount of a phenyl-quinoline-carboxylic acid pyridine derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### EXAMPLES

The invention is further illustrated with reference to the following examples, which are not intended to be in any way limiting to the scope of the invention as claimed.

### Example 1

### Preparatory Example

### Synthetic Route

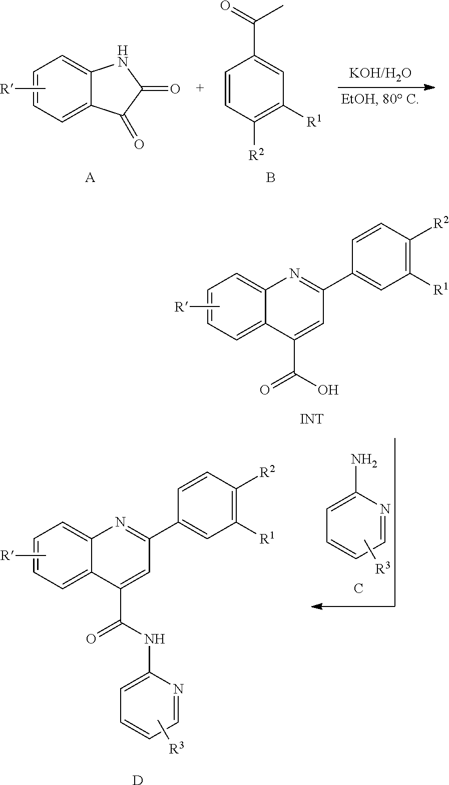

### Experimental Procedures

The synthetic pathway of the 2-phenyl-quinoline-4-carboxylic acid pyridin-2-ylamides (D) of the invention involves coupling between the suitably-prepared 2-phenyl-quinoline-4-carboxylic acid (INT) and a proper pyridin-2-ylamine (C). The pyridin-2-ylamines C, when not commercially-available, are synthesised by conventional methods. Amide formation is carried out upon treatment of the intermediate carboxylic acid (INT) with an excess of thionyl chloride to afford the corresponding acylchloride, which is reacted with the suitable pyridin-2-ylamine in presence of a tertiary base. It is preferred that this latter reaction is carried out overnight, in a mixture of dichloromethane and an excess of triethylamine, and at room temperature or under heating. The usual work-up consists of evaporation to dryness of the reaction mixture and the crude residue is then washed with HCl 1 N and water, and finally purified either by crystallization or flash chromatography. Alternatively, the compounds of the invention have been synthesized by mixing directly the intermediate carboxylic acids (INT) and pyridin-2-ylamines (C) in presence of a coupling reagent (1-propanephosphonic acid cyclic anhydride) and triethylamine. The reaction mixture, cooled upon addition of the reagents, is usually stirred at room temperature overnight, diluted with dichoromethane, washed with water, dried and evaporated to dryness, to afford the amides of the inventions. These latter are purified either by crystallization or column chromatography, and their purity (>96%) is evaluated at HPLC. In presence of methyl ether substituent(s), ether cleavage follows, upon mild nucleophilic substitution with the Lewis acid boron tribromide. The 2-phenyl-quinoline-4-carboxylic acid intermediates (INT) are obtained in high yields by the Pftizinger reaction of isatines (A) with the appropriate commercially-available arylketones (B) in 99% ethanol and aqueous potassium hydroxide solution (85%) at 80° C. for 1-2 days. The usual work-up consists of evaporation of the solvent, dissolution of the residue in water and washing with diethylether. The ice-cooled aqueous phase is acidified to pH 1 with 37% HCl and the solid precipitated is filtered, dried and purified either by crystallization or flash chromatography.

2-(3,4-Dichloro-phenyl)-quinoline-4-carboxylic acid (INT-1)

1H-Indole-2,3-dione (isatin; 2.000 g, 13.5932 mmol), 1-(3,4-dichloro-phenyl)-ethanone (3.084 g, 16.3118 mmol) and aqueous potassium hydroxide solution (85%; 40.7796 mmol) are dissolved in ethanol (99%; 20 ml) and the mixture is stirred at 80° C. for 18 hrs under a nitrogen atmosphere. The resulting reaction mixture is evaporated, the residue is dissolved in water (30 ml) and the solution is washed with diethylether (2×15 ml). The aqueous phase is ice cooled, acidified to pH 1 with 37% HCl (˜10 ml), and the red solid precipitated is filtered, washed with water and dried overnight (˜4.3 g). This crude residue is purified by crystallization form ethanol (2.200, ˜51% yield).

2-(4-Methoxy-phenyl)-quinoline-4-carboxylic acid (INT-2)

1H-Indole-2,3-dione (isatin; 4.000 g, 27.1864 mmol), 1-(3,4-dichloro-phenyl)-ethanone (4.695 g, 31.2644 mmol) and aqueous potassium hydroxide solution (85%; 81.5592 mmol) are dissolved in ethanol (99%; 30 ml) and the mixture is stirred at 80° C. for 20 hrs under a nitrogen atmosphere. The resulting reaction mixture is evaporated, the residue is dissolved in water (30 ml) and the solution is washed with diethylether (2×20 ml). The aqueous phase is ice cooled, acidified to pH 1 with 37% HCl (˜12 ml), and the red solid precipitated is filtered, washed with water and dried overnight (˜7.6 g). This crude residue is purified by crystallization form diethylether and hexane (7.000, ˜92% yield).

2-(3,4-Dimethoxy-phenyl)-quinoline-4-carboxylic acid (INT-3)

1H-Indole-2,3-dione (isatin; 4.000 g, 27.1864 mmol), 1-(3,4-dimethoxy-phenyl)-ethanone (5.634 g, 31.2644 mmol) and aqueous potassium hydroxide solution (85%; 81.5592 mmol) are dissolved in ethanol (99%; 30 ml) and the mixture is stirred at 80° C. for 20 hrs under a nitrogen atmosphere. The resulting reaction mixture is evaporated, the residue is dissolved in water (30 ml) and the solution is washed with diethylether (2×20 ml). The aqueous phase is ice cooled, acidified to pH 1 with 37% HCl (˜12 ml), and the red solid precipitated is filtered, washed with water and dried overnight (˜8.4 g). This crude residue is purified by crystallization form diethylether and hexane (7.700, ˜92% yield).

### Compounds of the Invention

2-(3,4-Dichloro-phenyl)-quinoline-4-carboxylic acid (6-fluoro-pyridin-2-yl)-amide (Compound 1)

To a stirred and ice-cooled solution of 2-(3,4-dichloro-phenyl)-quinoline-4-carboxylic acid (INT-1; 7.000 g, 22.0016 mmol) and 2-amino-6-fluoropyridine (2.467 g, 22.0016 mmol) in dichloromethane (200 ml), triethylamine (15.584 g, ˜21.5 ml, 154.0112 mmol) is added, followed by portion-wise addition of 1-propanephosphonic acid cyclic anhydride (37.803 g, 59.4043 mmol) and the mixture is allowed to attain room temperature spontaneously overnight. The resulting mixture is concentrated (˜100 ml), washed with water and brine, dried (MgSO₄), and evaporated to afford a brown gummy residue (9.100 g, 100% mass balance). This is purified by column chromatography eluting with 8% ethyl acetate in hexane, to obtain the title compound as an orange solid (0.865 g, 9.53% yield). M.p. 204.2-205.5° C.

2-(4-Chloro-phenyl)-quinoline-4-carboxylic acid (6-fluoro-pyridin-2-yl)-amide (Compound 2)

To a stirred and ice-cooled solution of commercial 2-(4-chloro-phenyl)-quinoline-4-carboxylic acid (1.400 g, 4.9346 mmol) and 2-amino-6-fluoropyridine (0.664 g, 5.9215 mmol) in dichloromethane (20 ml), triethylamine (3.495 g, ˜4.8 ml, 34.5422 mmol) is added, followed by portion-wise addition of 1-propanephosphonic acid cyclic anhydride (12.561 g, 19.7384 mmol) and the mixture is allowed to attain room temperature spontaneously overnight. The resulting mixture is diluted with dichloromethane (˜30 ml), washed with water and brine, dried (MgSO₄), and evaporated to afford a brown gummy residue (1.860 g, 100% mass balance). This is purified by column chromatography eluting with 8% ethyl acetate in hexane, to obtain the title compound as a pale yellow solid (0.474 g, ˜18% yield). M.p. 200-201° C.

2-(3,4-Dichloro-phenyl)-quinoline-4-carboxylic acid (5-fluoro-pyridin-2-yl)-amide (Compound 3)

To a stirred and ice-cooled solution of commercial 2-(3,4-dichloro-phenyl)-quinoline-4-carboxylic acid (INT-1; 3.000 g, 9.4293 mmol) and 5-fluoro-pyridin-2-ylamine (1.374 g, 12.2581 mmol) in dichloromethane (35 ml), triethylamine (6.679 g, ˜22.5 ml, 66.0051 mmol) is added, followed by portion-wise addition of 1-propanephosphonic acid cyclic anhydride (24.002 g, 37.7172 mmol) and the mixture is allowed to attain room temperature spontaneously overnight. The resulting mixture is diluted with dichloromethane (˜40 ml), washed with water and brine, dried (MgSO₄), and evaporated to afford a reddish gummy residue (3.900 g, 100% mass balance). This is purified by crystallization from chloroform and hexane, to obtain the title compound as a yellow solid (2.000 g, ˜55% yield). M.p. 203.6-205.0° C.

2-(4-Methoxy-phenyl)-quinoline-4-carboxylic acid (6-fluoro-pyridin-2-yl)-amide (Compound 4)

To a stirred and ice-cooled solution of 2-(4-methoxy-phenyl)-quinoline-4-carboxylic acid (INT-2; 2.500 g, 8.9512 mmol) and 6-fluoro-pyridin-2-ylamine (1.204 g, 10.7414 mmol) in dichloromethane (30 ml), triethylamine (6.340 g, ˜8.7 ml, 62.6584 mmol) is added, followed by portion-wise addition of 1-propanephosphonic acid cyclic anhydride (22.785 g, 35.8048 mmol) and the mixture is allowed to attain room temperature spontaneously overnight. The resulting mixture is diluted with dichloromethane (˜40 ml), washed with water and brine, dried (MgSO₄), and evaporated to afford a reddish gummy residue (3.350 g, 100% mass balance). This is purified by column chromatography eluting with 12-16% ethyl acetate in hexane, to obtain the title compound as a pale yellow solid (0.320 g, ˜10% yield). M.p. 174.4-175.7° C.

2-(3,4-Dimethoxy-phenyl)-quinoline-4-carboxylic acid (6-fluoro-pyridin-2-yl)-amide (Compound 5)

To a stirred and ice-cooled solution of 2-(3,4-dimethoxy-phenyl)-quinoline-4-carboxylic acid (INT-3; 2.000 g, 6.4658 mmol) and 6-fluoro-pyridin-2-ylamine (0.870 g, 7.759 mmol) in dichloromethane (20 ml), triethylamine (4.580 g, ˜15.4 ml, 45.2606 mmol) is added, followed by portion-wise addition of 1-propanephosphonic acid cyclic anhydride (16.458 g, 25.8632 mmol) and the mixture is allowed to attain room temperature spontaneously overnight and stirred at this temperature for additional 20 hours. The resulting mixture is diluted with dichloromethane (˜50 ml), washed with water and brine, dried (MgSO₄), and evaporated to afford a brown gummy residue (2.600 g, 100% mass balance). This is purified by column chromatography eluting with 32-36% ethyl acetate in hexane, to obtain the title compound as a pale yellow solid (0.300 g, ˜11% yield). M.p. 225.2-226.4° C.

2-(4-Hydroxy-phenyl)-quinoline-4-carboxylic acid (6-fluoro-pyridin-2-yl)-amide (Compound 6)

To a stirred solution of 2-(4-methoxy-phenyl)-quinoline-4-carboxylic acid (6-fluoro-pyridin-2-yl)-amide (Compound 5; 0.270 g, 0.7231 mmol) in anhydrous dichloromethane (15 ml), cooled to −78° C. and under a nitrogen flow, a solution of boron tribromide (1.268 g, ˜0.5 ml, 5.0617 mmol) in 4 ml of anhydrous dichloromethane is added drop wise. The mixture is allowed to reach room temperature spontaneously overnight and it is then cooled again in an ice-salt bath and the excess of the reagent is decomposed upon drop-wise addition of 2 ml of methanol and 5 ml of water. After 10 min stirring, 10% sodium hydroxide solution (5 ml) is added and the aqueous layer, once separated, is acidified with 10% hydrochloric acid solution and extracted with chloroform (3×40 ml). The combined organic layers are dried over MgSO₄, filtered and evaporated to afford a solid residue (˜0.26 g). This material, washed with petroleum ether and dried (0.210 g, 81% yield), is >96% pure at HPLC. M.p. 210.4-211.6° C.

In analogy herewith the following compounds were made:

2-(4-Bromo-phenyl)-quinoline-4-carboxylic acid (5-methyl-pyridin-2-yl)-amide (Compound 7)

M.p. 182.4-183.7° C. LC-ESI-HRMS of [M+H]+ shows 418.0547408 Da. Calc. 418.054955 Da, dev. −0.5 ppm.

8-Fluoro-2-(4-methoxy-phenyl)-quinoline-4-carboxylic acid (6-fluoro-pyridin-2-yl)-amide (Compound 8)

M.p. 201.8-202.8° C. LC-ESI-HRMS of [M+H]+ shows 392.1224731 Da. Calc. 392.120513 Da, dev. 5 ppm.

8-Fluoro-2-(4-hydroxy-phenyl)-quinoline-4-carboxylic acid (6-fluoro-pyridin-2-yl)-amide (Compound 9)

M.p. 254.0-256.0° C.

6-Fluoro-2-(4-methoxy-phenyl)-quinoline-4-carboxylic acid (6-fluoro-pyridin-2-yl)-amide (Compound 10)

M.p. 161.2-162.9° C.

6-Fluoro-2-(4-hydroxy-phenyl)-quinoline-4-carboxylic acid (6-fluoro-pvridin-2-yl)-amide (Compound 11)

M.p. 253.5-254.5° C. LC-ESI-HRMS of [M+H]+ shows 378.1048 Da. Calc. 378.104863 Da, dev. −0.2 ppm.

2-Benzo[1,3]dioxo-5-yl-quinoline-4-carboxylic acid (6-fluoro-pyridin-2-yl)-amide (Compound 12)

M.p. 135-138° C. LC-ESI-HRMS of [M+H]+ shows 388.1109 Da. Calc. 388.1092 Da, dev. 4.4 ppm.

2-(2,3-Dihydro-benzo[1,4]dioxin-6-yl)-quinoline-4-carboxylic acid (6-fluoro-pyridin-2-yl)-amide (Compound 13)

M.p. 209.9-210.6° C. LC-ESI-HRMS of [M+H]+ shows 402.1253 Da. Calc. 402.12485 Da, dev. 1.1 ppm.

7-Fluoro-2-(4-methoxy-phenyl)-quinoline-4-carboxylic acid (6-fluoro-pvridin-2-yl)-amide (Compound 14)

M.p. 188.4-189.9° C. LC-ESI-HRMS of [M+H]+ shows 392.1203 Da. Calc. 392.120513 Da, dev. −0.5 ppm.

### Example 2

### Biological Activity

In this example the positive modulation of wild-type nAChR α7 receptors by Compounds 4 and 5, representative of the invention, was determined using nAChR α7 receptors heterologously expressed in Xenopus laevis oocytes.

The electrical current through the nAChR α7 channel was measured using conventional two-electrode voltage clamp and nAChR α7 currents were activated by applying pulses of agonist-containing solution onto the nAChR α7 expressing oocyte.

In brief, the oocytes were placed in a recording chambers and continuously super-fused with an Oocyte Ringer (OR) solution containing 90 mM NaCl, 2.5 mM KCl, 2.5 mM CaCl₂, 1 mM MgCl₂ and 5 mM HEPES (pH adjusted to 7.4). The oocytes were clamped at −60 mV and currents were induced by applying 20 s pulses of 100 μM acetylcholine dissolved in OR. The intervals between the acetylcholine applications were 5 minutes, during which the oocytes were washed with OR. The first three applications were control applications to insure a constant response level of 100 μM acetylcholine. For the subsequent test applications, increasing concentrations (0.01-31.6 μM) of the test compound were applied 30 s before and during the acetylcholine (100 μM) application, which caused a robust increase in the acetylcholine-induced current amplitude.

The positive modulation in the presence of Compound 4 was calculated as (test-control)/control×100% and the concentration response curve for this positive modulation was fitted to the sigmoidal logistic equation: I=Iₘₐₓ/(1+(EC₅₀/[compound])ⁿ), where Iₘₐₓ represents the maximal modulation of the control response, EC₅₀ is the concentration causing a half maximal response, and n is the slope coefficient.

The calculated EC₅₀ values for Compounds 4 and 5, were 3.7 and 1.2 μM, respectively, and the calculated EC₅₀ Iₘₐₓ values for Compounds 4 and 5, were 283 and 197%, respectively. This is an indication of a biological activity as potent modulators of the nicotinic acetylcholine α7 receptor subtype.

1. A phenyl-quinoline-carboxylic acid pyridine derivative represented by Formula I 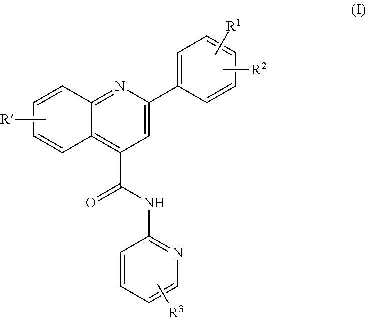
a stereoisomer thereof or a mixture of its stereoisomers, or a pharmaceutically acceptable salt thereof, wherein
R′ represents hydrogen or halo;
R¹ and R², independently of each other, represent a substituent selected from the group consisting of hydrogen, halo, trifluoromethyl, trifluoromethoxy, nitro, cyano, hydroxy and alkoxy; or
R¹ and R² together represents a methylenedioxy or ethylenedioxy group; and and
R³ represents hydrogen, halo, nitro and alkyl;
provided, however,
that R¹, R² and R³ cannot all represent hydrogen; and
if R¹ and R² both represent hydrogen, then R³ cannot represent chloro or nitro or methyl; and
if R¹ and R² both represent chloro or methoxy, then R³ cannot represent hydrogen, chloro, bromo, nitro or methyl; and
if one of R¹ and R² represents halo or alkoxy, and the other of R¹ and R² represents hydrogen, then R³ cannot represent hydrogen, chloro, nitro or methyl.

2. The phenyl-quinoline-carboxylic acid pyridine derivative of claim 1, a stereoisomer thereof or a mixture of its stereoisomers, or a pharmaceutically acceptable salt thereof, wherein R′ represents hydrogen or halo.

3. The phenyl-quinoline-carboxylic acid pyridine derivative of claim 1, a stereoisomer thereof or a mixture of its stereoisomers, or a pharmaceutically acceptable salt thereof, wherein
R¹ and R², independently of each other, represent a substituent selected from the group consisting of hydrogen, halo, trifluoromethyl, trifluoromethoxy, nitro, cyano, hydroxy and alkoxy; or
R¹ and R² together represent a methylenedioxy or ethylenedioxy group.

4. The phenyl-quinoline-carboxylic acid pyridine derivative of claim 1, a stereoisomer thereof or a mixture of its stereoisomers, or a pharmaceutically acceptable salt thereof, wherein R¹ and R², independently of each other, represent a substituent selected from the group consisting of hydrogen, halo, trifluoromethyl, trifluoromethoxy, nitro, cyano, hydroxy and alkoxy.

5. The phenyl-quinoline-carboxylic acid pyridine derivative of claim 1, a stereoisomer thereof or a mixture of its stereoisomers, or a pharmaceutically acceptable salt thereof, wherein R¹ and R² together represent a methylenedioxy or ethylenedioxy group.

6. The phenyl-quinoline-carboxylic acid pyridine derivative of claim 1, a stereoisomer thereof or a mixture of its stereoisomers, or a pharmaceutically acceptable salt thereof, wherein R³ represents hydrogen, halo, nitro or alkyl.

7. The phenyl-quinoline-carboxylic acid pyridine derivative of claim 1, which is
2-(3,4-Dichloro-phenyl)-quinoline-4-carboxylic acid (6-fluoro-pyridin-2-yl)-amide;
2-(4-Chloro-phenyl)-quinoline-4-carboxylic acid (6-fluoro-pyridin-2-yl)-amide;
2-(3,4-Dichloro-phenyl)-quinoline-4-carboxylic acid (5-fluoro-pyridin-2-yl)-amide;
2-(4-Methoxy-phenyl)-quinoline-4-carboxylic acid (6-fluoro-pyridin-2-yl)-amide;
2-(3,4-Dimethoxy-phenyl)-quinoline-4-carboxylic acid (6-fluoro-pyridin-2-yl)-amide;
2-(4-Hydroxy-phenyl)-quinoline-4-carboxylic acid (6-fluoro-pyridin-2-yl)-amide;
2-(4-Bromo-phenyl)-quinoline-4-carboxylic acid (5-methyl-pyridin-2-yl)-amide;
8-Fluoro-2-(4-methoxy-phenyl)-quinoline-4-carboxylic acid (6-fluoro-pyridin-2-yl)-amide;
8-Fluoro-2-(4-hydroxy-phenyl)-quinoline-4-carboxylic acid (6-fluoro-pyridin-2-yl)-amide;
6-Fluoro-2-(4-methoxy-phenyl)-quinoline-4-carboxylic acid (6-fluoro-pyridin-2-yl)-amide;
6-Fluoro-2-(4-hydroxy-phenyl)-quinoline-4-carboxylic acid (6-fluoro-pyridin-2-yl)-amide;
2-Benzo[1,3]dioxo-5-yl-quinoline-4-carboxylic acid (6-fluoro-pyridin-2-yl)-amide;
2-(2,3-Dihydro-benzo[1,4]dioxin-6-yl)-quinoline-4-carboxylic acid (6-fluoro-pyridin-2-yl)-amide; or
7-Fluoro-2-(4-methoxy-phenyl)-quinoline-4-carboxylic acid (6-fluoro-pyridin-2-yl)-amide;
a stereoisomer thereof or a mixture of its stereoisomers, or a pharmaceutically acceptable salt thereof.

8. A pharmaceutical composition comprising a therapeutically effective amount of a phenyl-quinoline-carboxylic acid pyridine derivative of claim 1, a stereoisomer thereof or a mixture of its stereoisomers, or a pharmaceutically acceptable addition salt thereof, together with at least one pharmaceutically acceptable carrier or diluent.

9. (canceled)

10. (canceled)

11. A method of treatment, prevention or alleviation of a disease or a disorder or a condition of a living animal body, including a human, which disorder, disease or condition is responsive to modulation of nicotinic acetylcholine receptors, which method comprises the step of administering to such a living animal body in need thereof a therapeutically effective amount of a phenyl-quinoline-carboxylic acid pyridine derivative of claim 1, a stereoisomer thereof or a mixture of its stereoisomers, or a pharmaceutically acceptable addition salt thereof.

12. The method according to claim 11, wherein the disease, disorder or condition responsive to modulation of nicotinic acetylcholine receptors is anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, peripheral neuropathy, autism, dyslexia, tardive dyskinesia, hyperkinesia, epilepsy, post-traumatic syndrome, social phobia, a sleeping disorder, pseudo dementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhoea, asthma, epilepsy, tardive dyskinesia, hyperkinesia, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to postherpetic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated with withdrawal symptoms caused by termination of use of addictive substances, including nicotine withdrawal symptoms, opioid withdrawal symptoms, including heroin, cocaine and morphine, benzodiazepine withdrawal symptoms including benzodiazepine-like drugs and alcohol.
